(19) United States
(12) Patent Application Publication
(10) Pub. No.: US 2025/0174337 A1
(43) Pub. Date: May 29, 2025

(54) ARTIFICIAL INTELLIGENCE-BASED PERSONALIZED PREDICTIVE TREATMENT SYSTEM

(71) Applicant: Hsin-Yin Chiang, Strasbourg (FR)

(72) Inventor: Hsin-Yin Chiang, Strasbourg (FR)

(73) Assignee: Cephalgo SAS, Strasbourg (FR)

(21) Appl. No.: 18/519,180

(22) Filed: Nov. 27, 2023

(51) Int. Cl. G16H 20/70; A61B 5/00; A61B 5/372; G16H 10/60; G16H 40/67; G16H 50/20

(52) U.S. Cl. CPC G16H 20/70; A61B 5/0006; A61B 5/372; G16H 10/60; G16H 40/67; G16H 50/20

(57) ABSTRACT

The present invention discloses an artificial intelligence-based (or AI-based) system and method for providing personalized treatment plans to individuals suffering from mental health disorders. By leveraging EEG measurements, patient data, and AI algorithms, this system addresses the unique needs of each patient and enhances the overall quality of mental health care. The AI-based personalized treatment system has the potential to revolutionize mental health care. By amalgamating EEG measurements and an extensive range of patient-specific factors, it enables a tailored, dynamic approach to mental health treatment that takes into account the complete patient profile. The integration of this system into existing electronic health record (EHR) systems or patient monitoring platforms creates a patient-centered experience that enhances treatment outcomes, patient satisfaction, and reduces the strain on healthcare professionals.

### BACKGROUND OF THE INNOVATION

### A. Technical Field

The invention falls within the field of healthcare, particularly in the domain of mental health treatment. The present invention relates to an artificial intelligence-based (or AI-based) system for providing personalized treatment plans to individuals suffering from mental health disorders. The system utilizes EEG (electroencephalogram) measurements along with various patient-specific factors to create tailored treatment strategies encompassing medication, psychotherapy, and other therapeutic interventions.

### B. Description of Related Art

Mental health disorders pose a significant global health challenge, impacting individuals' quality of life, productivity, and overall well-being. The complexity and variability of these disorders demand a personalized treatment approach that encompasses the diverse factors contributing to each patient's condition. The current invention addresses this critical need by introducing an innovative and comprehensive AI-based personalized treatment system that utilizes EEG measurements and a multitude of patient-specific factors to revolutionize mental health care.

Some of known prior arts tried to solve this problem in a way as follows, U.S. Pat. No. 10,325,070 of Live Network Inc., discloses a computerized system for and method of providing precision healthcare services such as consultation, education, assessment, diagnosis, intervention, or treatment at a distance via encrypted real-time image and audio presence where the healthcare professional's assessment, diagnosis, and intervention activities are informed by patient feedback, smart objects, and artificial intelligence and patient outcomes are optimized through recursive system feedback. Another known prior art, U.S. Pat. No. 10,617,351 of Sackett Solutions and Innovations LLC, discloses methods and systems to periodically monitor the emotional state of a subject comprising the steps of: exposing the subject to a plurality of stimuli during a session; acquiring objective data from a plurality of monitoring sensors, wherein at least one sensor measures a physiological parameter; transferring the data to a database, and processing the data to extract objective information about the emotional state of the subject.

However, above-mentioned traditional methods and systems of treating mental health disorders often rely on standardized treatment plans that may not fully consider the unique characteristics of each patient. This one-size-fits-all approach can lead to suboptimal outcomes and prolonged suffering for individuals. The invention seeks to overcome these limitations by harnessing the power of EEG measurements, which provide insights into a patient's brain activity, and integrating them with a wide range of patient-related factors to create personalized treatment plans that adapt dynamically over time. The invention's core concept involves utilizing EEG measures to tailor treatment plans from a diverse set of therapeutic options. These options include medication categories beyond the conventional antidepressants and mood stabilizers, encompassing antipsychotics, anti-anxiety medications, stimulants, and other specialized medications depending on the specific mental health disorder. Furthermore, psychotherapy options, such as cognitive behavioral therapy (CBT), dialectical behavior therapy (DBT), psychodynamic therapy, and other specific methods, are considered. Additionally, alternative therapies like ITMS, ECT, Vagus Nerve Stimulation (VNS), and psychoeducation are included to offer a comprehensive range of treatment possibilities.

Crucially, the personalized treatment plans are not solely based on EEG measurements but are developed by considering a rich array of patient-specific factors. These factors encompass demographic attributes, cultural considerations, medical history, severity of symptoms, co-occurring psychiatric or medical conditions, response to prior treatments, potential for substance abuse, suicidal tendencies, cognitive function, living conditions, and support networks. This holistic approach aims to create treatment plans that reflect the intricacies of each patient's life circumstances and challenges, leading to improved treatment outcomes.

To achieve this groundbreaking innovation, a database is established that houses the multifaceted patient factors, EEG measurements, and treatment outcomes. Artificial intelligence algorithms are employed to analyze and learn from this data by using EEG signal as the objective metric to classify the treatment outcomes together with other patient-specific factors, enabling the creation of personalized treatment plans. The AI system adapts to each patient's profile by comparing their attributes and EEG measurements with the database, thus ensuring treatment plans are continually refined and optimized.

The invention also embraces real-time monitoring using EEG devices, whether in a laboratory or as wearable devices for continuous remote monitoring. For patients utilizing wearable EEG devices, the AI system can detect significant changes in EEG patterns, worsening symptoms, signs of self-harm, adverse medication effects, and suggest treatment plan adjustments, ensuring timely intervention and patient safety.

Furthermore, the AI system monitors daily life factors that impact mental health, including sleep quality, physical activity, social interactions, and substance use. This comprehensive approach addresses the interconnected nature of mental health and allows for more accurate treatment adaptations.

Therefore, there is a need for an AI-based personalized treatment system outlined in this invention has the potential to revolutionize mental health care. By amalgamating EEG measurements and an extensive range of patient-specific factors, it enables a tailored, dynamic approach to mental health treatment that takes into account the complete patient profile. The integration of this system into existing electronic health record (EHR) systems or patient monitoring platforms creates a patient-centered experience that enhances treatment outcomes, patient satisfaction, and reduces the strain on healthcare professionals. This innovation represents a significant step forward in the field of mental health care, offering hope for more effective, holistic, and personalized interventions.

### SUMMARY OF THE INNOVATION

The present invention discloses an artificial intelligence-based (or AI-based) system for providing personalized treatment plans to individuals suffering from mental health disorders. The system comprises, a database comprising patient-specific factors, including age, gender, pathology, culture, medical history, symptom severity, comorbid conditions, biological test result, mental health assessment, treatment responses, cognitive function, living situation, and support network. The system further comprises, an electroencephalogram (EEG) measurement module configured to acquire EEG measurements from a patient.

Further, the system comprises, an AI-based treatment generation module employing a set of adaptive algorithms, said algorithms utilizing said patient-specific factors to generate a personalized treatment plan from a plurality of treatment options. In this context, the treatment response refers to the comprehensive annotation encompassing the entire input, which includes EEG data and patient-specific factors, used for training the aforementioned AI-based treatment generation module. Artificial intelligence algorithms are employed to analyze and learn from this data by using EEG signal as the objective metric to classify the treatment outcomes together with other patient-specific factors, enabling the creation of personalized treatment plans. The treatment options may include medication, psychotherapy, and other therapies. The system comprises, an evaluation module configured to receive patient evaluation results from medical professionals, patients, and patient's family, said results comprising standardized assessment scores and psychotherapy inputs. The system further comprises, an EEG pattern comparison module configured to compare new patient EEG measurements with stored EEG measurements in the database, thereby tailoring the treatment plan.

In one embodiment, the system comprises a notification module configured to transmit alerts to medical professionals based on detected changes in EEG patterns, worsening symptoms, suicidal ideation, or adverse medication effects. The system comprises, a daily life monitoring module for tracking sleep, physical activity, social interactions, and substance use of patients. The system further comprises, a treatment plan adjustment module adapting the treatment plan based on real-time factors including EEG measurements and daily life data. The treatment plan adjustment module is an AI learning module, configured to learn from mistakes and inquire the AI-based treatment generation module to calibrate in accordance to an output result, if the suggestion is not effective

In some embodiments, the system retrieves inputs of the EEG measurement module and other information, and suggests the suitable treatment for the patient. the other information includes: patient medication history, medical history, questionnaires such as patient health questionnaire (PHQ-9) and generalized anxiety disorder assessment (GAD-7), cognitive assessment, and other biologics testing information including, inflammatory biomarkers and growth factors, metabolomic analysis, transcriptomic analysis, epigenomic analysis, pharmacogenetic and long QT phenotype, hormonal/cortisol analysis, immunoprofiling, and all the possible tests.

In various embodiments, the EEG pattern comparison module is configured to compare the patient's EEG measurements with stored EEG measurements in the database and suggests the suitable treatment for the patient. The stored EEG measurements is EEG measurements of other patients. The EEG measurement module is configured to acquire one or more EEG measurements continuously from a patient while receiving other treatments. In one embodiment, the system is configured to provide any one or both of a diagnosis and a prognosis suggestion. The system is configured to provide one or more information of the possible side-effects in accordance to the patient's one or more EEG measurements. The system is also used together or integrated with the existing remote patient monitoring system to adjust the treatment according to the EEG measurements, if changed.

According to another embodiment of the present invention, a method for generating personalized mental health treatment plans, is disclosed. The method comprises the steps of: (1) acquiring EEG measurements from a patient using EEG measurement devices; (2) gathering patient-specific factors, including age, gender, pathology, culture, medical history, symptom severity, comorbid conditions, treatment responses, cognitive function, living situation, and support network; (3) comparing new patient EEG measurements with stored EEG measurements in a database to refine the treatment plan; (4) utilizing an AI-based treatment generation module employing adaptive algorithms to process said patient-specific factors and generate a personalized treatment plan from a plurality of treatment options, comprising medication, psychotherapy, and other therapies; (5) receiving patient evaluation results from medical professionals, patients, and patient's family, by the AI learning module, said results comprising standardized assessment scores and psychotherapy inputs; (6) transmitting alerts to medical professionals based on detected EEG pattern changes, worsening symptoms, suicidal ideation, or adverse medication effects; (7) monitoring daily life factors, including sleep, physical activity, social interactions, and substance use, and (8) adapting the treatment plan based on real-time factors including EEG measurements and daily life data. In some embodiments, the sequence of first and second step can be reversed, depending on the admission process of each clinic.

Other objects, features and advantages of the present innovation will become apparent from the following detailed description. It should be understood, however, that the detailed description and the specific examples, while indicating specific embodiments of the innovation, are given by way of illustration only, since various changes and modifications within the spirit and scope of the innovation will become apparent to those skilled in the art from this detailed description.

### BRIEF DESCRIPTION OF DRAWINGS

The foregoing summary, as well as the following detailed description of the innovation, is better understood when read in conjunction with the appended drawings. For the purpose of illustrating the innovation, exemplary constructions of the innovation are shown in the drawings. However, the innovation is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates an environment of a personalized predictive treatment system, according to an embodiment of the present invention.

FIG. 2 exemplarily illustrates another environment of a personalized predictive treatment system, according to an embodiment of the present invention.

FIG. 3 exemplarily illustrates yet another environment of a personalized predictive treatment system, according to an embodiment of the present invention.

FIG. 4 exemplarily illustrates a personalized predictive treatment system, according to an embodiment of the present invention.

FIG. 5 exemplarily illustrates a flowchart of a method for generating personalized mental health treatment plans, according to an embodiment of the present invention.

FIG. 6 exemplarily illustrates a flowchart for overall monitoring over patients (with different time scales) to well document the treatment effectiveness for the personalized prediction system, according to an embodiment of the present invention.

### DETAILED DESCRIPTION OF EMBODIMENTS

A description of embodiments of the present innovation will now be given with reference to the Figures. It is expected that the present innovation may be embodied in other specific forms without departing from its spirit or essential characteristics. The described embodiments are to be considered in all respects only as illustrative and not restrictive.

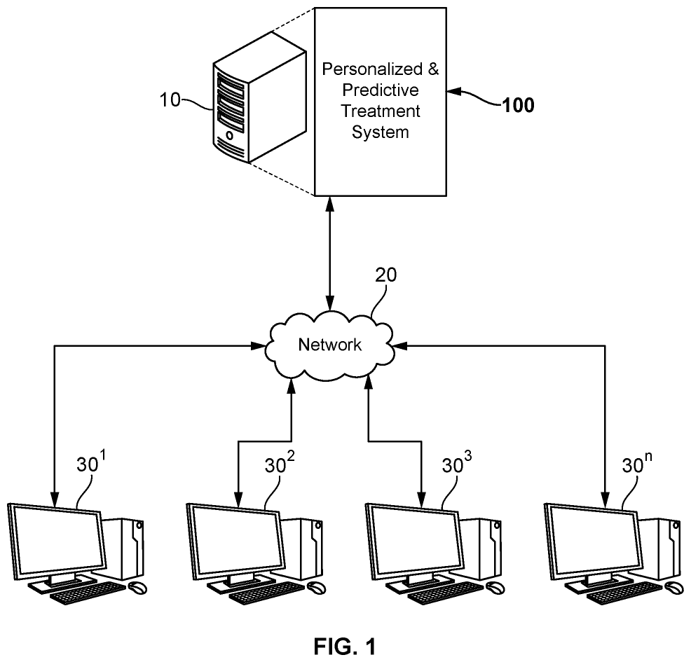

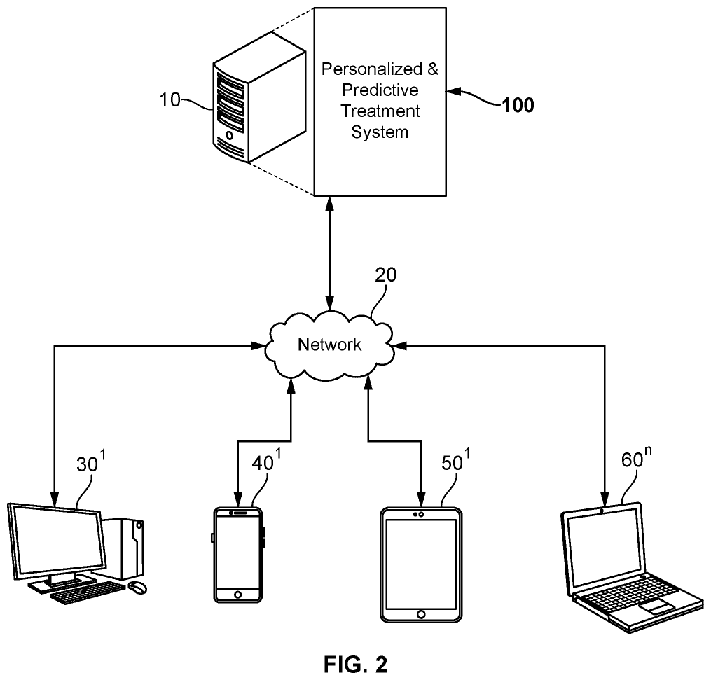

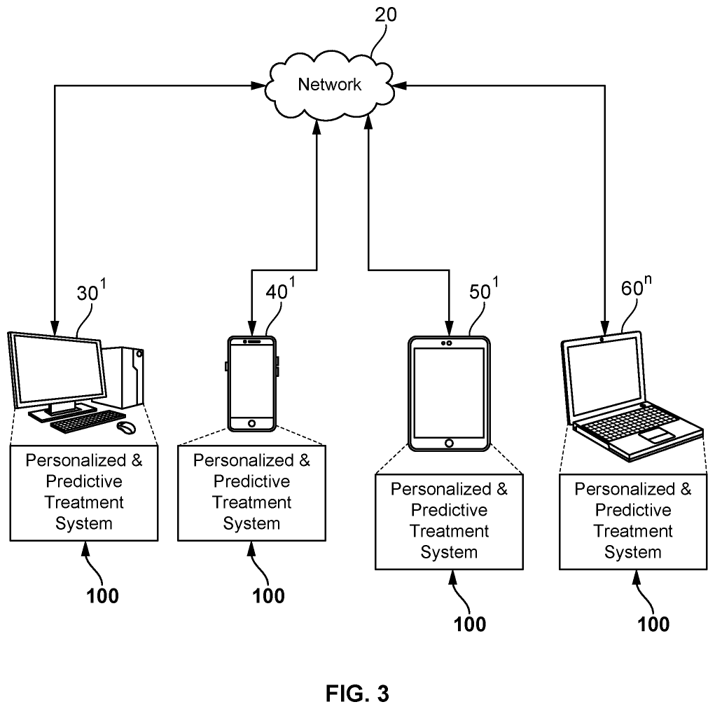

The present invention relates to an innovative AI-based system designed to deliver personalized treatment plans for individuals afflicted with mental health disorders. The system combines electroencephalogram (EEG) measurements, patient-specific factors, adaptive algorithms, and real-time monitoring to create tailored interventions. FIG. 1 exemplarily illustrates an environment of an artificial intelligence (AI) based system 100 designed to deliver personalized treatment plans for individuals afflicted with mental health disorders, according to an embodiment of the present invention. The environment illustrated in FIG. 1, includes a personalized and predictive treatment system 100 hosted in server 10 in communication with one or more user devices (30¹, 30², 30³ . . . , 30ⁿ) associated with one or more users, respectively, via network 20. FIG. 2 exemplarily illustrates another environment of an artificial intelligence (AI) based system 100, wherein the one or more user devices could be a desktop computer (30¹), a mobile phone or a smart phone (40¹), a tablet or a palmtop (50¹), or a laptop (60ⁿ). FIG. 3 exemplarily illustrates yet another environment of an artificial intelligence (AI) based system 100, wherein the system 100 is hosted directly in any user devices, but not limited to, desktop computer (30¹), a mobile phone or a smart phone (40¹), a tablet or a palmtop (50¹), or a laptop (60ⁿ).

In some embodiments, the user device (30¹, 30², 30³ . . . , 30ⁿ) is a computing device configured to provide access to the service provided by the server 10. The user device (30¹, 30², 30³ . . . , 30ⁿ) is configured to provide an interface to access the services provided by the server 10. The interface, for example, is an application that allows the user device (30¹, 30², 30³ . . . , 30ⁿ) to wirelessly connect with the server 10 via the network 20. The user device (30¹, 30², 30³ . . . , 30ⁿ) is a communication device. The user device (30¹, 30², 30³ . . . , 30ⁿ) may be, for example, a desktop computer, a laptop computer, a mobile phone, a personal digital assistant, and the like.

In some embodiments, the server 10 could be any suitable server(s) for storing information, data, programs, and/or any other suitable content. In an example, the server 10 is at least one of a general or special purpose computer. The server 10 operates as a single computer, which could be a hardware and/or software server, a workstation, a desktop, a laptop, a tablet, a mobile phone, a mainframe, a supercomputer, a server farm, and so forth. Although the server 10 is illustrated as a single device, the functions performed by server 10 could be performed using any suitable number of computing devices. In some embodiments, the network 20 generally represents one or more interconnected networks, over which the user device (30¹, 30², 30³ . . . , 30ⁿ) and the server 10 could communicate with each other. The network 20 may include packet-based wide area networks (such as the Internet), local area networks (LAN), private networks, wireless networks, satellite networks, cellular networks, paging networks, and the like. A person skilled in the art will recognize that the network 20 may also be a combination of more than one type of network. For example, the network 20 may be a combination of a LAN and the Internet. In addition, the network 20 may be implemented as a wired network or a wireless network or a combination thereof.

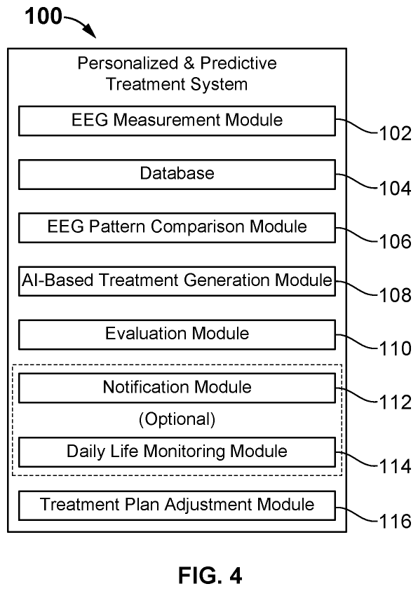

FIG. 4 exemplarily illustrates an artificial intelligence-based (or AI-based) system 100 for providing personalized treatment plans to individuals suffering from mental health disorders, according to an embodiment of the present invention. In one embodiment, the system 100 comprises, an electroencephalogram (EEG) measurement module 102, a database 104, an EEG pattern comparison module 106, an AI-based treatment generation module 108, an evaluation module 110, a notification module 112, a daily life monitoring module 114, and a treatment plan adjustment module 116. In another embodiment, the system 100 may include any new modules in future, for providing personalized treatment plans to individuals suffering from mental health disorders.

In one embodiment, the system 100 includes an EEG measurement module 102 responsible for acquiring EEG measurements from patients. This module 102 can employ EEG measurement devices, which are capable of capturing the electrical activity within the brain. These measurements provide valuable insights into the patient's neurological and mental states and form a foundation for the subsequent personalized treatment plan. The EEG measurement module is configured to acquire one or more EEG measurements continuously from a patient while receiving other treatments using one or more EEG devices. EEG measurements are acquired while the patient is in either an awake or a sleeping state, providing a comprehensive assessment of brain activity across varying levels of consciousness. The EEG measurement module 102 follows a detailed algorithm to ensure accurate data collection and analysis. During the data acquisition phase, EEG data is collected using an EEG cap or electrodes placed on the scalp, with the data sampled at an appropriate rate, such as 256 Hz or higher. In the preprocessing phase, a bandpass filter is applied to remove noise outside the desired frequency range, typically 0.5-50 Hz. Artifacts such as eye blinks, muscle movements, and other non-brain signals are removed using Independent Component Analysis (ICA) or other artifact removal techniques such as algorithms trained by Convolutional Neural Network or Deep Neural Network. The data is then re-referenced to a common average reference or another suitable reference. In the epoching phase, the continuous EEG data is divided into epochs or segments based on the experimental design or analysis needs, with epochs containing excessive noise or artifacts being removed.

EEG devices are specialized equipment designed to measure and record electrical activity in the brain. They consist of several essential components, (a) Electrodes: EEG electrodes are sensors that are placed on the scalp to detect electrical signals produced by brain cells (neurons). These electrodes are typically attached using a conductive gel or paste to ensure good electrical conductivity. (b) Amplifiers: EEG amplifiers amplify the weak electrical signals detected by the electrodes to levels suitable for further processing and analysis. They enhance the signal-to-noise ratio, making it easier to identify and interpret brainwave patterns. (c) Data Acquisition System: This system includes the hardware and software responsible for capturing and recording the amplified electrical signals from the electrodes. It digitizes the analog signals into a digital format for easier storage, analysis, and visualization. (d) Signal Processing and Analysis Software: EEG data collected by the system is processed and analyzed using specialized software. This software can filter and segment the data, extract features, and generate visual representations like spectrograms, power spectra, and event-related potentials (ERPs).

There are different types of EEG Devices, (1) Conventional EEG Machines: These are traditional EEG machines used in clinical settings. They typically involve a cap or a series of individual electrodes attached to the scalp. The signals from these electrodes are transmitted to a centralized amplifier and data acquisition system for recording and analysis. (2) Ambulatory EEG Devices: These are portable EEG devices that allow for longer-term monitoring outside of a clinical setting. Ambulatory EEGs are often used to monitor brain activity for extended periods, which is particularly useful in diagnosing conditions like epilepsy. (3) Wireless EEG Devices: Some modern EEG devices are wireless, eliminating the need for cumbersome wires and allowing for greater mobility and comfort for the individual being monitored. Wireless EEG systems transmit data to the acquisition unit without physical cables. (4) Dry Electrode EEG Systems: Traditional EEG systems use wet electrodes with conductive gel. In contrast, dry electrode EEG systems use electrodes that do not require gel application. These are more convenient and less messy, making them increasingly popular in research and clinical applications. EEG devices are sophisticated tools comprising electrodes, amplifiers, data acquisition systems, and specialized software. They come in various forms and have diverse applications in clinical diagnosis, research, brain-computer interfaces, and therapeutic interventions.

In one embodiment of the present invention, EEG device is anyone of in-built in the system, portable device for outside clinic use, or in-clinic use. In-Built EEG device option, the EEG device is integrated or built-in within the system, such as AI-based predictive treatment system 100. This could be a specialized system designed for data acquisition, processing, and analysis, where the EEG component is an integral part. For instance, in a research or clinical setting, there might be a dedicated system that includes the EEG hardware and software, allowing for seamless EEG recordings and analysis within a specific environment. In portable EEG Device for outside clinic option, an EEG device that is designed to be portable, allowing for EEG recordings to be taken outside of a clinical or laboratory setting. Portable EEG devices are lightweight, compact, and usually battery-powered, enabling data collection in a variety of settings such as homes, ambulatory settings, or field research. This is particularly useful for long-term monitoring or studying brain activity in real-world scenarios. At In-clinic EEG device option, a standard EEG device typically found in a clinical or hospital setting. These devices are often more sophisticated, offering advanced features for precise EEG recordings, monitoring, and analysis. In-clinic EEG devices are usually stationary and may be connected to a network for centralized data storage and analysis. They are commonly used for diagnostic purposes, medical research, or monitoring patients during various medical procedures. Each option has its own set of advantages and use cases. The choice of EEG device depends on the specific requirements of the study, research, or clinical application, considering factors such as mobility, data quality, ease of use, and the intended environment for EEG data collection and analysis.

The invention incorporates a database 104 where the EEG data may come from the previous measurement and the EEG measurement made from EEG measurement module 102. This database 104 further contains an array of patient-specific factors as a collective label for the corresponding EEG measurement. These factors encompass diverse elements such as age, gender, cultural background, medical history, severity of symptoms, biological test result, mental health assessment, comorbid conditions, responses to prior treatments, cognitive function, living environment, and support network which have different weights for the following data analysis. In various embodiments, the present invention requires all data related to EEG, treatment responses, and other patient-specific factors. This comprehensive repository enables the AI-based system to holistically understand each patient's unique circumstances.

To refine the personalized treatment plan, the system 100 further integrates an EEG pattern comparison module 106. This module analyzes EEG data to optimize treatment plans. This module 106 compares the new EEG measurements acquired from the patient with EEG measurements stored in the database 104 from the same patient and/or from other patients. The EEG pattern comparison module 106 employs a comprehensive system that involves several key steps in the process of comparing patient EEG data and other relevant factors. While specific methods and formulations may vary, the following general steps are typically considered: (a) Feature Extraction: Relevant features are extracted from the EEG data, which may include power spectral density and event-related potentials. (b) Power Envelope Calculation: The power envelope of the EEG signal is determined, often by techniques like the Hilbert transform and amplitude squaring. (c) Connectivity Analysis: Connectivity analysis is conducted by measuring correlation or coherence between the power envelopes of different EEG channels, with statistical tests to assess the significance of these measures. (d) Visualization: Results from connectivity analysis are visualized using connectivity maps or graphs. (e) Patient Profiling: A patient profile is created for each individual, incorporating EEG features and patient-specific factors like age, gender, cultural background, medical history, severity of symptoms, biological test result, mental health assessment, comorbid conditions, responses to prior treatments, cognitive function, living environment, and support network which have different weights for the following data analysis. (f) Normalization and Standardization: Data is normalized and standardized to ensure optimized feature weighting during comparison. (g) Distance Metric Calculation: A distance metric, such as Euclidean distance or cosine similarity, is calculated between the profile vector of each patient and all others in the database to measure similarity or dissimilarity. (h) Ranking and Filtering: Patients are ranked based on the distance metric, with a threshold set to filter out dissimilar patients. Further filtering is often performed based on specific criteria like matching pathology or medication history. (i) Results Visualization: Results are visualized using suitable graphs, such as scatter plots or dendrograms. (j) Report Generation: A report is generated, listing the most similar patients, their distance metrics, and patient-specific factors. (k) Validation and Improvement: Results are validated by comparing predicted matches with ground truth data, if available. Feedback from clinicians and experts is incorporated to refine the algorithm and enhance its accuracy. These steps provide a systematic approach to comparing EEG patterns and other patient-specific factors to identify similar patients in a database. The specific algorithms and techniques used can vary, but this general process ensures a holistic comparison that considers both neurological and clinical aspects of each patient.

By analyzing changes in EEG patterns, the system 100 optimizes the treatment plan, adapting it to the patient's evolving neurological and mental states. In other words, the EEG pattern comparison module 106 is configured to compare the patient's EEG measurements with stored EEG measurements in the database 104 and the stored EEG measurements are EEG measurements of other existing or already treated patients. A central aspect of the present invention is the AI-based treatment generation module 108. Treatment recommendation generation module 108 may consist of three parts, treatment recommendation generation process, cross validation, and monitoring and feedback. Treatment recommendation generation process begins with the input of the target patient's EEG patterns from EEG measurement module 102 and other patient-specific factors. Relevant features are extracted from the EEG data from the database 104, such as power spectral density and event-related potentials.

Machine learning algorithms are then applied to analyze these features in conjunction with the patient-specific factors. A database query is performed to access historical data of previous patients, their EEG patterns, patient-specific factors, and treatment responses. Similarity matching algorithms, such as cosine similarity or Euclidean distance, are used to compare the target patient's profile with those in the database. Based on the similarity scores, effective treatments for patients with similar profiles are retrieved. The output is a list of recommended treatments for the target patient. In the cross-validation for treatment recommendation process, we start by gathering all necessary inputs, including the list of recommended treatments, the patient's medical history, allergies, current medications, and any other relevant factors. A safety check is then performed, which involves accessing a database containing information about drug interactions, contraindications, and known allergies. Treatment interaction analysis is conducted for each recommended treatment to check if it interacts with any of the patient's current medications. Allergy and contraindication checks are also performed by cross-referencing the recommended treatments with the patient's known allergies and reviewing the patient's medical history to identify any conditions that might contraindicate the recommended treatments. The compatibility check ensures that combining the recommended treatments won't result in synergistic effects that could harm the patient and that the patient isn't already on a medication that serves the same purpose as the recommended treatment. Based on the results of the cross-validation, any treatments found to be unsafe are removed from the recommendation list, and the remaining treatments are prioritized based on their likelihood of being effective and causing the least side effects or interactions.

Before finalizing the recommendations, Lastly, the monitoring and feedback step involves continuously collecting feedback from the medical professional at first and patient and the family in the later stage in the evaluation module 110. Techniques and tools such as Database Management Systems (DBMS), Decision Trees or Rule-Based Systems, and Collaborative Filtering can be employed to streamline and enhance the process. The resulting treatment recommendation is tailored from a range of treatment options, including medication, psychotherapy techniques, and various other therapies. The AI's capacity to adapt the algorithms according to the specifics of each patient ensures that the treatment recommendations are finely tuned.

The evaluation module 110 is a comprehensive process that begins with data collection, where initial evaluation results are gathered from medical professionals to validate the resulting treatment recommendation, and once the new treatment is applied, evaluation results are also collected from patients themselves and the patient's family. Every collected data will be entered in the database 104. The evaluations encompass standardized assessment scores, which provide quantifiable metrics for gauging the patient's condition. Furthermore, psychotherapy inputs, including emotional states, behaviors, and coping strategies, contribute to the AI's understanding of the patient's psychological landscape. The collected data will then be preprocessing, which involves standardizing the data format and units for analysis and handling any missing or incomplete data. Feature extraction follows, where relevant features such as specific symptoms, emotional states, behaviors, and coping strategies are extracted from the evaluation results. In the data analysis phase, quantitative analysis is performed on the standardized assessment scores to quantify the patient's condition, and natural language processing (NLP) algorithms are used for qualitative analysis to extract insights into the patient's emotional states, behaviors, and coping strategies. The initial evaluation results from medical professionals are then used to validate the resulting treatment recommendation generated from treatment recommendation generation module 108. In case the treatment recommendation is not validated by the medical professional, iterations from the AI-based treatment generation module 108 will be made from the medical professional's feedback. Once the treatment recommendation is adapted, the patient profile will be updated in database 104 with the new treatment plan and new evaluation results and insights.

In cases, where the patient employs wearable EEG devices for continuous monitoring, the system 100 encompasses a notification module 112. This module 112 is designed to send alerts to medical professionals when notable changes in EEG patterns are detected. Alerts can also be triggered by indicators such as worsening symptoms, signs of suicidal ideation, or potential adverse effects of medication. The notification module 112 is a structured process that starts with continuous data collection, where EEG data is continuously monitored and collected from wearable devices for home use or on-site devices at clinics, with differentiation between various types of EEG data such as sleep monitoring, conscious monitoring (cognitive EEG, emotional EEG, sensory EEG, motor EEG, etc.), resting-state EEG, event-related potentials (ERPs), and ambulatory EEG, along with additional patient-specific data like but not limited to symptoms, medication effects, and psychotherapy inputs. The collected data will be also entered in database 104. In the data preprocessing phase, the data format and units are standardized for analysis, any missing or incomplete data is handled, and EEG data is separated based on its type (sleep or conscious) and source (home or clinic). Feature extraction follows, where relevant features are extracted from the EEG data, considering the type of EEG (sleep or conscious) and the source of data (home or clinic), along with features from additional patient data. Pattern recognition utilizes machine learning algorithms to analyze EEG patterns and detect any notable changes, identify patterns associated with worsening symptoms, signs of suicidal ideation, or potential adverse effects of medication, and differentiate between normal patterns for sleep EEG and those indicative of a problem. Alert generation then generates alerts based on the identified patterns, customizes alerts based on the severity and urgency of the situation, and considers the source of EEG data (home or clinic) and the type of EEG (sleep or conscious) when generating alerts. In the notification sending phase, alerts are sent to medical professionals through various communication channels such as but not limited to email, SMS, or in-app notifications, with an interface provided for medical professionals to review the patient's progression directly through the evaluation module in cases where EEG measurement takes place on-site at clinics, and alerts sent to both medical professionals and the patient or their family in cases where EEG measurement takes place at home. Finally, the Feedback Loop collects feedback from medical professionals, patients, and their families on the accuracy and relevance of the alerts, and uses this feedback to improve the alert generation and notification sending processes. In cases, where the EEG measurement takes place on-site with the medical professionals, the professionals can review the patient's progression directly through the evaluation module 110.

Additionally, the system 100 features a daily life monitoring module 114. The daily life monitoring module 114 follows a structured algorithm to effectively track and analyze a patient's daily life factors, adapt their treatment plan accordingly, and provide valuable insights to both the patient and medical professionals. In this module, the patient's wearable device data, mobile application data, and self-reported data are gathered, including sleep quality, physical activity, social interactions, and substance use. This comprehensive dataset is then output to the database 104 and initiates continuous improvement of personalized treatment. This real-time data enriches the system's understanding of the patient's overall well-being and augments treatment adaptability. An integral component of the present invention is the treatment plan adjustment module 116. This module 116 dynamically adapts the treatment plan based on real-time factors. These factors include newly acquired EEG measurements, current daily life data, and updates from the patient evaluation results. By incorporating up-to-date information, the system ensures the treatment remains responsive to the patient's evolving needs. In the treatment plan module 116, the comprehensive database 104, the result from evaluation module 110, notification module 112, and daily life monitoring module 114, and the patient's current treatment plan from AI-Based Treatment Generation Module 108 are analyzed to identify any discrepancies or areas where the treatment plan may no longer be effective or optimal. A report detailing the analysis results and any identified discrepancies is then generated. Following this, the AI Learning and Calibration step utilizes AI learning algorithms to learn from any mistakes or inefficiencies in the current treatment plan. The AI-based Treatment Generation Module 108 is then inquired to calibrate the treatment plan according to the output result. If the suggested treatment is not effective, the treatment plan is adjusted accordingly, resulting in a calibrated and optimized treatment plan for the patient. In the final step, Treatment Plan Adaptation, the calibrated treatment plan is adapted to the patient's current condition and needs, considering any new information or changes in the patient's condition, and resulting in an adapted and personalized treatment plan for the patient. In one aspect, the treatment plan adjustment module 116 is an AI learning module, configured to learn from mistakes and inquire the AI-based treatment generation module 108 to calibrate in accordance to an output result, if the suggestion is not effective

In one embodiment, the system 100 retrieves inputs of the EEG measurement module 102 and other information, and suggests the suitable treatment for the patient. It also considers additional medical data and assessments which includes patient medication history, medical history, questionnaires such as patient health questionnaire (PHQ-9) and generalized anxiety disorder assessment (GAD-7), cognitive assessment, and other biologics testing information including, inflammatory biomarkers and growth factors, metabolomic analysis, transcriptomic analysis, epigenomic analysis, pharmacogenetic and long QT phenotype, hormonal/cortisol analysis, immunoprofiling, and all other possible tests. In one embodiment, the system 100 is configured to provide any one or both of a diagnosis and a prognosis suggestion. The system 100 is configured to provide one or more information of the possible side-effects in accordance to the patient's one or more EEG measurements. The system 100 is also integrated with the existing remote patient monitoring system to adjust the treatment according to the EEG measurements, if changed.

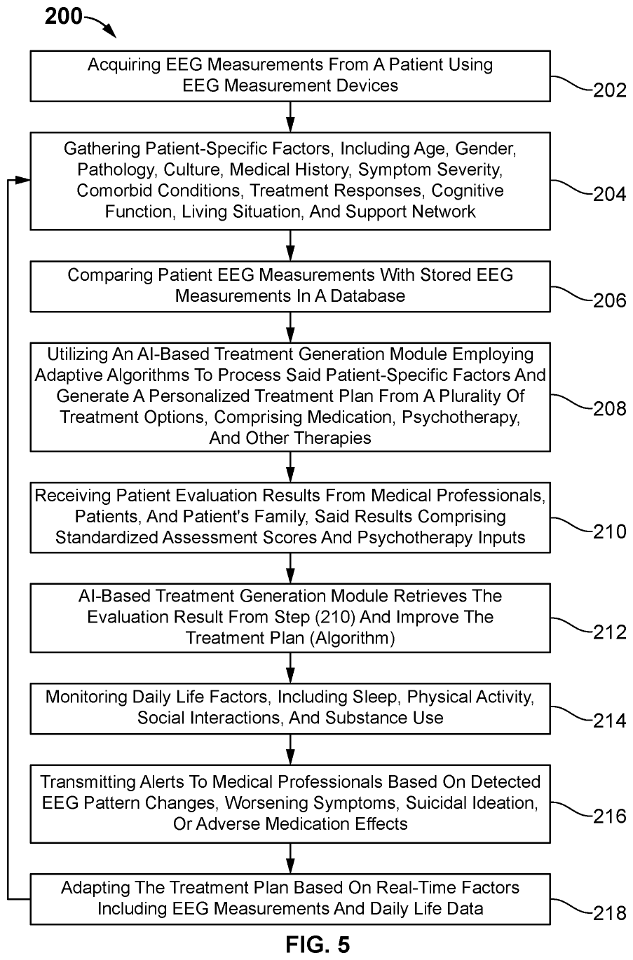

According to another embodiment of the present invention, a method 200 for generating personalized mental health treatment plans, is disclosed. This method involves a series of steps that utilize both patient-specific data and AI algorithms to create a tailored treatment plan. Artificial intelligence algorithms are employed to analyze and learn from this data by using EEG signal as the objective metric to classify the treatment outcomes together with other patient-specific factors, enabling the creation of personalized treatment plans. FIG. 5 exemplarily illustrates a flowchart of the method 200 for generating personalized mental health treatment plans, according to an embodiment of the present invention. The method 200 comprises the steps of: (202) acquiring EEG measurements from a patient using EEG measurement devices; (204) gathering patient-specific factors, including age, gender, cultural background, medical history, severity of symptoms, biological test result, mental health assessment, comorbid conditions, responses to prior treatments, cognitive function, living environment, and support network which have different weights for the following data analysis; (206) comparing new patient EEG measurements with stored EEG measurements in a database to refine the treatment plan; (208) utilizing an AI-based treatment generation module employing adaptive algorithms to process said patient-specific factors and generate a personalized treatment plan from a plurality of treatment options, comprising medication, psychotherapy, and other therapies; (210) receiving patient evaluation results from medical professionals, patients, and patient's family, said results comprising standardized assessment scores and psychotherapy inputs; (212) transmitting alerts to medical professionals based on detected EEG pattern changes, worsening symptoms, suicidal ideation, or adverse medication effects; (214) monitoring daily life factors, including sleep, physical activity, social interactions, and substance use, and (216) adapting the treatment plan based on real-time factors including EEG measurements and daily life data. In certain instances, there is a flexibility to interchange the sequence of steps 202 and 204 to suit the particular context. Additionally, depending on specific requirements, optional modules 214 and 216 can be either incorporated into the process or excluded.

In the present invention, various embodiments are disclosed, each involving distinct iteration loops. These iterations are instrumental in executing specific processes or algorithms outlined within the present invention. For instance, one such iteration is delineated from step 218 to step 204, which plays a pivotal role in the operation of the described system. It should be noted that the iterations are an essential component of the disclosed inventions, providing the necessary framework for achieving the intended objectives of the present invention. These iterations may vary in terms of scope, duration, and purpose, and they are all considered integral to the overall functionality and innovation presented herein.

In some embodiments, each AI module may be trained by the following steps but not limited to: (1) Data Preprocessing: The raw data is cleaned and formatted; (2) Database Augmentation: New patient data is continuously added; (3) Model Training: Suitable machine learning algorithms are selected and trained; (4) Hyperparameter Tuning: The model parameters are optimized; (5) Model Validation: The model's performance is assessed; (6) Model Interpretability: The model's decision-making is analyzed; (7) Model Deployment: The trained model is integrated into the system.

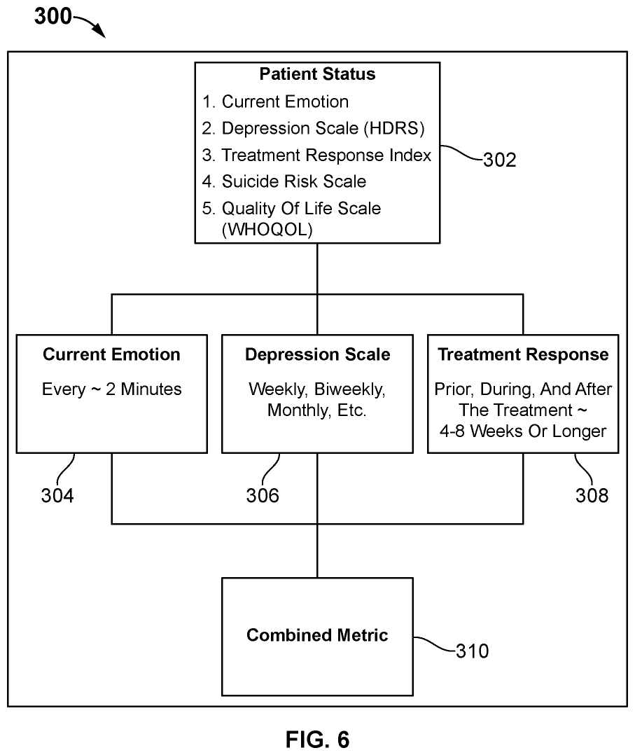

FIG. 6 exemplarily illustrates an example for overall monitoring over patients (with different time scales) to well document the treatment effectiveness for the personalized prediction system 100, according to an embodiment of the present invention. Various information or monitoring data is taken into account such as, but not limited to, Patient Status 302, Current Emotion 304, Depression Scale 306, and/or Treatment Response 308. In the embodiments with continuous EEG monitoring, Current Emotion 304 documents the emotion variation in a unit of seconds and the emotion classification is derived by triggers including, but not limited to those listed in FIG. 6. Each trigger is designed to trigger the designated emotion which is used as the label to train AI to recognize emotions via EEG measurements. Furthermore, the said AI also learns to distinguish the outliers in terms of emotion-labeled EEG or patients if the consistent irregularity has been observed and inform the professionals. In larger time scales such as two weeks, the patients are asked to document their Depression Scale 306 via designated questionnaires with or without the admission of medical professionals. This Depression Scale 306 will then be used to train AI to recognise the patient's depression level by analyzing EEG measurement. Furthermore, a patient's Treatment Response 308 is typically observed in a time scale of 8-12 weeks prior to the treatment, during the treatment, and after the treatment. The assessments about treatment response conducted by the medical professionals are then used to train AI to learn the treatment effect via EEG.

The collective of AI models enable to provide combined metric 310 (Inputs—from different AI algorithms and Outputs—defining the patient's status). Henceforth, the present invention addresses the complex nature of mental health disorders and aims to provide tailored interventions that consider the entirety of the patient's circumstances, symptoms, medical history, and responses to various treatments. The system 100 employs advanced AI algorithms to analyze the collected data and generate optimal treatment plans, thereby enhancing patient outcomes and reducing the burden on healthcare professionals.

Personalized Treatment Options: The inventive system 100 offers personalized treatment plans for mental health disorders by utilizing EEG measurements and considering various treatment modalities. The following options are assessed and tailored to each patient's unique profile: Medication: The system 100 evaluates a range of medications, including antidepressants, mood stabilizers, antipsychotics, anti-anxiety medications, stimulants, and other relevant drugs based on the specific mental health disorder. Psychotherapy: Different psychotherapy approaches such as cognitive behavioral therapy (CBT), dialectical behavior therapy (DBT), interpersonal psychotherapy (IPT), psychodynamic therapy, Eye Movement Desensitization and Reprocessing (EMDR), and Exposure and Response Prevention (ERP) for OCD are considered based on the patient's condition. Other Therapies: The system 100 also accounts for treatments like repetitive transcranial magnetic stimulation (rTMS), electroconvulsive therapy (ECT), Vagus Nerve Stimulation (VNS), Deep Brain Stimulation (DBS), Light Therapy, and psychoeducation.

Factors Considered for Personalization: The inventive system 100 takes into account a comprehensive set of patient-specific factors when generating personalized treatment plans: Patient Profile: Age, gender, cultural background, medical history, severity of symptoms, biological test result, mental health assessment, comorbid conditions, response to previous treatments, potential for substance abuse, suicidal ideation, cognitive function, living situation, and support network. Professional and Patient Feedback: Inputs from medical professionals, patients, and their families, including treatment effectiveness, side effects, cognitive function assessments (e.g., MMSE or MoCA), and disorder-specific assessments (e.g., Y-BOCS, MADRS, HAM-D, YMRS, CAPS). Patient Self-Evaluation: Utilization of patient self-assessment tools such as PHQ-9, GAD-7, BDI, and others to gain insight into the patient's emotional and mental state. Psychotherapy Inputs: Capturing emotional state, behaviors, thoughts, environment, coping strategies, support system, stressors, physiological responses, and past experiences to tailor psychotherapy interventions.

AI-Driven Personalization: The inventive system 100 features a robust AI framework that incorporates a database of patient profiles and corresponding treatment outcomes. The AI algorithms continuously adapt and evolve based on this dataset. When a new patient profile is introduced, the AI algorithm compares the patient's characteristics to the database, thereby generating a personalized treatment plan that optimally matches the patient's profile. EEG Measurements and Continuous Monitoring: The system 100 accommodates EEG measurements obtained either within a laboratory or through wearable devices for continuous monitoring. In the latter case, the AI is capable of detecting significant changes in the patient's EEG patterns, heightened anxiety or depressive symptoms, signs of suicidal ideation or self-harm, and potential adverse medication side effects. Notifications regarding these changes can be sent to medical professionals, prompting necessary treatment adjustments.

Comprehensive Data Integration: The AI monitors various aspects of the patient's daily life that influence mental health, including sleep quality, physical activity, social interactions, and substance use. This holistic approach ensures that treatment plans evolve based on real-time patient experiences and behaviors. Integration with Existing Systems: The entire system 100 is designed to seamlessly integrate with Electronic Health Record (EHR) systems and other patient monitoring platforms. This facilitates the sharing of information among healthcare professionals, providing a comprehensive view of the patient's treatment journey.

In one embodiment, the system 100 is a computing device integrated with Artificial Intelligence (AI) modules. Artificial Intelligence (AI) modules refer to distinct components or specialized software components within the system 100 that perform specific tasks or functions. These modules are designed to handle particular aspects of data processing, analysis, decision-making, and interaction within the system 100. They work together to enable the system 100 to perform complex tasks, learn from data, and generate intelligent outputs. Each module serves a specific purpose and contributes to the overall functionality of the system 100.

The system 100 further have the capability to provide the user an interface to interact with the services provided by the server. The interface, for example, a mobile application that allows the system 100 to wirelessly connect with the server via the network. The system 100 connect with the server via Bluetooth by scanning a QR code. Further, the system 100 could be connected with the server via any other known methods. The system 100 is configured to execute one or more client applications such as, without limitation, a web browser to access and view content over a computer network, an email client to send and retrieve emails, an instant messaging client for communicating with other users, and a File Transfer Protocol (FTP) client for file transfer. The system 100 in various embodiments, may include a Wireless Application Protocol (WAP) browser or other wireless or mobile device protocol suites. In some embodiments, the system 100 is hosted directly in any one or more server, or any one or more user device itself. In some embodiments, the system 100 is a computer-readable medium storing instructions for executing personalized and predictive treatment plans.

The network generally represents one or more interconnected networks, over which the computing device and the server can communicate with each other. The network may include packet-based wide area networks (such as the Internet), local area networks (LAN), private networks, wireless networks, satellite networks, cellular networks, paging networks, and the like. A person skilled in the art will recognize that the network may also be a combination of more than one type of network. For example, the network may be a combination of a LAN and the Internet. In addition, the network may be implemented as a wired network, or a wireless network or a combination thereof.

In one embodiment, the server is at least one of a general or special purpose computer. In an embodiment, it operates as a single computer, which can be a hardware and/or software server, a workstation, a desktop, a laptop, a tablet, a mobile phone, a mainframe, a supercomputer, a server farm, and so forth. In an embodiment, the computer could be touchscreen and/or non-touchscreen device and could run on any type of OS, such as iOS™, Windows™, Android™, Unix™, Linux™ and/or others. In an embodiment, the computer is in communication with network. Such communication can be via a software application, a mobile app, a browser, an OS, and/or any combination thereof.

Preferred embodiments of this innovation are described herein, including the best mode known to the innovators for carrying out the innovation. It should be understood that the illustrated embodiments are exemplary only and should not be taken as limiting the scope of the innovation.

The foregoing description comprises illustrative embodiments of the present innovation. Having thus described exemplary embodiments of the present innovation, it should be noted by those skilled in the art that the within disclosures are exemplary only, and that various other alternatives, adaptations, and modifications may be made within the scope of the present innovation. Merely listing or numbering the steps of a method in a certain order does not constitute any limitation on the order of the steps of that method. Many modifications and other embodiments of the innovation will come to mind to one skilled in the art to which this innovation pertains having the benefit of the teachings in the foregoing descriptions. Although specific terms may be employed herein, they are used only in generic and descriptive sense and not for purposes of limitation. Accordingly, the present innovation is not limited to the specific embodiments illustrated herein.

What is claimed is:

1. A personalized predictive treatment system for mental health disorders, comprising:
an EEG measurement module configured to acquire one or more EEG measurements from a patient;
a database comprising one or more patient-specific factors;
an EEG pattern comparison module configured to compare patient EEG measurements with stored EEG measurements in the database;
an AI-based treatment generation module includes a set of adaptive algorithms, said algorithms utilizes said patient-specific factors to generate a personalized treatment plan from a plurality of treatment options;
an evaluation module configured to receive patient evaluation results from medical professionals, patients, and patient's family, said results comprising standardized assessment scores and psychotherapy inputs, and
a treatment plan adjustment module configured to adapt the treatment plan based on real-time factors including EEG measurements and daily life data.

2. The system of claim 1, wherein the database further comprises one or more patient-specific factors, including age, gender, pathology, culture, medical history, symptom severity, comorbid conditions, biological test result, mental health assessment, treatment responses, cognitive function, living situation, and support network.

3. The system of claim 1, wherein the treatment options include medication, psychotherapy, and other therapies.

4. The system of claim 1, wherein the system retrieves inputs of the EEG measurement module and other information, and suggests the suitable treatment for the patient.

5. The system of claim 4, wherein the other information includes: patient medication history, medical history, questionnaires such as patient health questionnaire (PHQ-9) and generalized anxiety disorder assessment (GAD-7), cognitive assessment, and other biologics testing information including, inflammatory biomarkers and growth factors, metabolomic analysis, transcriptomic analysis, epigenomic analysis, pharmacogenetic and long QT phenotype, hormonal/cortisol analysis, and immunoprofiling.

6. The system of claim 1, wherein the EEG pattern comparison module is configured to compare the patient's EEG measurements with stored EEG measurements in the database and suggests the suitable treatment for the patient, and wherein the stored EEG measurements is EEG measurements of other patients.

7. The system of claim 1, wherein the system is configured to provide any one or both of a diagnosis and a prognosis suggestion.

8. The system of claim 1, wherein the EEG measurement module is configured to acquire one or more EEG measurements continuously from a patient while receiving other treatments.

9. The system of claim 1, wherein the treatment plan adjustment module is an AI learning module, configured to learn from mistakes and inquire the AI-based treatment generation module to calibrate in accordance to an output result, if the suggestion is not effective.

10. The system of claim 1, wherein the system is configured to provide one or more information of the possible side-effects in accordance to the patient's one or more EEG measurements.

11. The system of claim 1, wherein the system is also integrated with the existing remote patient monitoring system to adjust the treatment according to the EEG measurements, if changed.

12. The system of claim 1, further comprises a notification module, wherein the notification module is configured to transmit alerts to medical professionals based on detected changes in EEG patterns, worsening symptoms, suicidal ideation, or adverse medication effects.

13. The system of claim 1, further comprises a daily life monitoring module, wherein the daily life monitoring module is configured to track daily life data including sleep, physical activity, social interactions, and substance use of patients.

14. A method generating personalized mental health treatment plans using a personalized predictive treatment system, comprising steps of:
(a) acquiring electroencephalogram (EEG) measurements from a patient using EEG measurement devices, via an EEG measurement module;
(b) gathering patient-specific factors, via a database;
(c) comparing, via an EEG pattern comparison module, patient EEG measurements with stored EEG measurements in a database;
(d) utilizing an AI-based treatment generation module employing adaptive algorithms to process said patient-specific factors and generate a personalized treatment plan from a plurality of treatment options, comprising medication, psychotherapy, and other therapies;
(e) receiving, via an evaluation module, patient evaluation results from medical professionals, patients, and patient's family, said results comprising standardized assessment scores and psychotherapy inputs;
(f) transmitting alerts, via a notification module, to medical professionals based on detected EEG pattern changes, worsening symptoms, suicidal ideation, or adverse medication effects;
(g) monitoring, via a daily life monitoring module, daily life factors, including sleep, physical activity, social interactions, and substance use, and
(h) adapting the treatment plan, via a treatment plan adjustment module, based on real-time factors including EEG measurements and daily life data.

15. The method of claim 14, wherein the database further comprises one or more patient-specific factors, including age, gender, pathology, culture, medical history, symptom severity, biological test result, mental health assessment, comorbid conditions, treatment responses, cognitive function, living situation, and support network.

16. The method of claim 14, wherein the EEG pattern comparison module is configured to compare the patient's EEG measurements with stored EEG measurements in the database and suggests the suitable treatment for the patient, and wherein the stored EEG measurements is EEG measurements of other patients.

17. The method of claim 14, wherein the EEG measurement module is configured to acquire one or more EEG measurements continuously from a patient while receiving other treatments using one or more EEG devices.

18. The method of claim 14, wherein the treatment plan adjustment module is an AI learning module, configured to learn from mistakes and inquire the AI-based treatment generation module to calibrate in accordance to an output result, if the suggestion is not effective.

19. The method of claim 17, wherein the EEG measurements are acquired while the patient is in either an awake or a sleeping state, providing a comprehensive assessment of brain activity across varying levels of consciousness.

20. The method of claim 17, wherein the EEG device is anyone of in-built in the system, portable device for outside clinic use, or in-clinic use.
